(19) United States
(12) Patent Application Publication
(10) Pub. No.: US 2002/0002153 A1
(43) Pub. Date: Jan. 3, 2002

(54) PHARMACEUTICAL PREPARATIONS FOR TREATING SIDE EFFECTS DURING AND/OR AFTER GNRHA THERAPY

(76) Inventors: MICHAEL OETTEL, JENA (DE); LUDWIG WILDT, HERZOGENAURACH-HAUNDORF (DE); PETER LICHT, BUBENREUTH (DE); JOACHIM NEUWINGER, ERLANGEN (DE); WOLFGANG HUMMEL, NEUENMARKT (DE); RALPH DITTRICH, ERLANGEN (DE)

Correspondence Address: STRIKER STRIKER AND STENBY, 103 EAST NECK ROAD, HUNTINGTON, NY, 11743

(21) Appl. No.: 09/291,385

(22) Filed: Apr. 14, 1999

Related U.S. Application Data
(60) Provisional application No. 60/081,791, filed on Apr. 15, 1998.

(51) Int. Cl. A61K 31/56

(52) U.S. Cl. 514/169; 514/170; 514/178; 514/182

(57) ABSTRACT

The pharmaceutical preparations for treating side effects, such as hot flashes, during and/or after treatment with analogs or antagonists of gonadotropin releasing hormone (GnRHa therapy) contain an effective amount of 17α-estradiol, its chemically modified derivatives, chemically modified derivatives of 17β-estradiol or estriol.

### CROSS-REFERENCE

This application claims the benefit of U.S. Provisional Patent Application Ser. No. 60/081,791, filed Apr. 15, 1998.

### BACKGROUND OF THE INVENTION

1. Field of the Invention

The present invention relates to pharmaceutical preparations for treating side effects, such as hot flashes, during and/or after treatment with analogs or antagonists of gonadotropin releasing hormone (GnRHa therapy).

2. Prior Art

It is known that treatment with analogs or antagonists of gonadotropin-releasing hormone (GnRHa therapy) is an effective therapy in diseases whose intensity is influenced by the activity of the gonads.

Examples of such diseases are endometriosis, benign genital disorders, filbroid tumors of the uterus, endometrial hyperplasia, premenstrual syndrome, catamenial epilepsies, and in the broader sense all cycle-dependent disorders in women, as well as carcinoma of the prostate in men.

According to A. E. Schindler, Der Frauenarzt [The Gynecologist] 2:211-214, 1995, from 8 to 12% of women of reproductive age suffer from endometriosis.

The principal symptoms of endometriosis, along with dysmenorrhea, are dyspareunia as well as cyclical and acyclical pain in the lower abdomen. It is also associated with problems of sterility and infertility.

Now that endometriosis has been identified as a progressive estrogen-dependent disease, attempts have been made to determine, besides surgical correction, what the most effective possible hormone therapy might be. To that end, a regimen for inducing pseudopregnancy (W. C. Andrews and G. D. Larsen, Am. J. Obst. Gynecol. 118(5):643-651, 1974), progestational hormones (K. S. Moghissi, J. Obstet. Gynecol. 47:265-267, 1976) and later Danazol (K. W. Schweppe, Endometriose [Endometriosis] 5:4-11) have primarily been employed. Today, GNRH analogs are available for inducing a reversible, medication-induced hypoestrogenism (P. A. Regidor, et al, Zentralbl. Gynädkol. [Central Gynecology Newsletter] 118:283-290, 1996).

The GNRH analogs used, such as Goserelin or Leuprorelin, slow down the development of gonadotropin in the pituitary gland and thus stop estrogen production in the ovaries and lead to amenorrhea, among other effects. They must either be injected once a month or administered twice daily as a nasal spray. The side effects of the GNRH analogs are similar to the complaints that occur during-menopause, such as hot flashes, sweating, headaches and depression.

Hot flashes and sweating are among the most unpleasant side effects of GnRHa therapy, which is also used in men for advanced and/or metastatic prostate cancer and thus is widely used. As a treatment that slows down hormonal action, GnRHa therapy is a palliative therapeutic principle. The quality of life of the patient during the treatment is given particular weight. According to S. Kliesch et al, Dtsch. Med. Wschr. [German Medical Weekly] 122:94-945, 1997, the incidence of hot flashes and the attendant outbreaks of sweating is a frequent and undesired accompanying symptom in up to 80% of female patients.

Acomparable incidence of hot flashes in men is described as being 58% after orchiectomy and 63% in therapy with GNRH analogs (A. V. Kaisary et al, Brit. J. Urol. 67:502-508, 1991).

To prevent hot flashes in general, until the present moment, for both women and men estrogens are the means of choice. In menopause and post-menopause, they have gained wide use in therapy for menopause-related complaints.

International Patent Disclosure WO 97/21704 discloses novel compounds as GNRH antagonists, among others in combination with estrogens, including therapy for hot flashes.

In GNRH analog therapy, the use of estrogens is not advisable, because the fundamental disease being treated is an estrogen-dependent disease (G. S. Dizerga et al, Fertil. Steril. 33:649-653, 1980) and would aggravate the clinical picture is estrogens were given.

Even in men, with GnRHa therapy for carcinoma of the prostate, the conventional administration of estrogens should be considered with restraint.

The undesired side effects that occur in estrogen therapy are exhibited among other ways in metabolic effects, which for instance lead to an increase in the binding globulins for sexual steroids, thyroid hormones, and cortisone. They are expressed as breast tenderness, headache, fluid retention, nausea, weight gain, and depression (R. Jewelewicz, Fertil. Steril. 67:1-12, 1997).

In men undergoing estrogen therapy, cardiovascular complications can also be expected as an important side effect (R. Haapianen et al, Brit. J. Urol. 66:94-97, 1990; R. Stege et al, Urologie [Urology] 34:398-403, 1995). Feminizing effects are also possible.

### SUMMARY OF THE INVENTION

The object of the invention is to discover novel pharmaceutical preparations with high effectiveness for treating side effects, such as hot flashes, during and/or after a treatment with analogs or antagonists of gonadotropin releasing hormone (GnRHa therapy).

The pharmaceutical preparations according to the invention for treating side effects, such as hot flashes, during and/or after treatment with analogs or antagonists of gonadotropin releasing hormone, contain at least one active ingredient selected from the group comprising 17α-estradiol, its chemically modified derivatives, or chemically modified derivatives of 17β-estradiol or estriol. The discovery of these novel preparations has attained the above set forth object of the invention.

Advantageous embodiments of the pharmaceutical preparations of the invention include the following particular active ingredients:

17α-estradiol

ent-17α-estradiol

4-methylestra-1,3,5(10)-triene-1,17α-diol

4-methylestra-1,3,5(10),6-tetraene-1,17α-diol

4-methylestra-1,3,5(10),6,8-pentaene-1,17α-diol

ent-estradiol

4-methylestra-1,3,5(10),6-tetraene-1,17β-diol

4-methylestra-1,3,5(10),6,8-pentaene-1,17β-diol

17α-4′-hydroxyphenylmethyl-4-methylestra-1,3,5(10)-triene-1,17β-diol

17α-4′-hydroxyphenoxymethyl-4-methylestra-1,3,5(10)-triene-1,17β-diol

17α-4′-hydroxythiophenoxymethyl-4-methylestra-1,3,5(10)-triene-1,17β-diol

17α-4′-dimethylaminophenylmethyl-4-methylestra-1,3,5(10)-triene-1,17β-diol

17α-3′,5′-dimethyl-4-hydroxyphenylmethyl-4-methylestra-1,3,5(10)-triene-1,17β-diol

17α-3′,5′-dimethyl-4′-hydroxyphenylmethyl-4-methylestra-1,3,5(10),6-tetraene-1,17β-diol

17α-4′-hydroxyphenoxylmethyl-4-methylestra-1,3,5(10),-tetraene-1,17β-diol

14β,15β-methylene-8-dehydroestradiol

6-dehydroestriol

9(11)-dehydroestriol.

The pharmaceutical preparations according to the invention include preparations for oral and parenteral administration, including topical, rectal, subcutaneous, intravenous, intramuscular, intraperitoneal, intranasal, intravaginal, intrabuccal, or sublingual administration, which along with typical vehicle and diluent agents include at least one of the active ingredients recited above or in the claims appended hereinbelow.

The forms of the administered preparations according to the invention include those given by mouth, such as tablets, capsules and lozenges or solutions; percutaneous preparations, such as transdermal therapeutic systems (TTSS) or gels, sprays or salves; intranasal preparations, such as a nasal spray or nose drops; rectal preparations, such as suppositories, and parenteral preparations, such as implants, pills, and ampoules. The various embodiments of the administrated preparations are prepared with the usual solid or liquid vehicles and diluents and the typically used pharmaceutical industry adjuvants, in accordance with the desired type of administration, in suitable dosages, in the known way. The advantages of the invention are attained essentially because pharmaceutical preparations have been discovered which prevent the conventional side effects, such as hot flashes, during and/or a treatment with analogs or antagonists of gonadotropin releasing hormone (GnRHa therapy) both in women and in men.

The same is true for side effects in other possible hormone-suppressing therapies.

### CLINICAL STUDY EXAMPLE

### 1. Methods

The symptoms displayed were investigated in the context of a clinical study.

The effectiveness of injected Decapeptyl-Depot (Ferring Arzneimittel GmbH, Kiel, Germany) in a dose of 3.2 mg of Triptorelin intramuscularly at intervals of four weeks and 1 to 2 mg of 17α-estradiol daily, given in the form of a vaginal suppository of 1 mg of 17α-estradiol each, was tested for 14 test subjects aged 22 to 38, who were being treated with Decapeptyl-Depot for endometriosis for at least six months.

Test subjects aged 22 to 46 with fibroid tumors of the uterus were given the same doses.

The treatment with 17α-estradiol was begun four weeks after the beginning of GnRHa therapy, that is, at the time of the second Decapeptyl injection.

Hot flashes were recorded by the subjects daily on a control form laid out for a 24-hour day, beginning with the first day of Decapeptyl therapy.

The mean number of hot flashes per 24 hours in weeks 3 and 4 of the GnRHa treatment (that is, before the beginning of the 17α-estradiol therapy) was assumed to be 100%, for the sake of better comparability. The number of hot flashes during additional therapy with 17α-estradiol was indicated as a percentage of the starting value. For five subjects, a measurement of skin temperature over 24 hours was done, for the sake of objectivity.

At intervals of four weeks, immediately prior to the injection of Decapeptyl-Depot, blood was drawn to determine LH, FSH, prolactin, testosterone, DHEAS, SHBG, free testosterone, estradiol, and progesterone. Blood specimens were also taken for determining a simple blood count, SGOT, SGPT, creatinine, Na, K, Ca, albumin, glucose and CA 125.

For long-term treatment, bone density was monitored at intervals of 6 to 12 months. In seven subjects, trials of withdrawal from 17α-estradiol over 4 to 8 weeks each were done.

### 2. Results of the Study

For all subjects, hot flashes occurred within four weeks after the onset of GnRHa therapy: on average, fourteen hot flashes per 24 hours; range: 7 to 18. The rise in skin temperature on average was 2.2° C.

Within one to three weeks after the first Decapeptyl-Depot injection, bleeding ensued in 12 subjects. In all the subjects, the LH and FSH values dropped below 3 I.U./ml after four weeks. Soon estradiol was on average 20 pg/ml; range:<15 pg/ml to 35 pg/ml.

Results of Administration of 17α-estradiol The number of hot flashes dropped significantly (p≦0.05, in a paired T test).

The drop could be found within one week and reached a stable level not later than after three weeks (see table 1).

TABLE 1
Hot flashes as a percentage of the starting value, with
17α-estradiol treatment

| | WEEK: | | |
|---|---|---|---|
| | 1^{st} | 2^{nd} | 3^{rd} |
| Subject No. 01 | 75% | 65% | 15% |
| Subject No. 02 | 70% | 60% | 30% |
| Subject No. 03 | 65% | 55% | 20% |
| Subject No. 04 | 79% | 40% | 15% |
| Subject No. 05 | 80% | 50% | 10% |
| Subject No. 06 | 90% | 40% | 10% |
| Subject No. 07 | 85% | 55% | 20% |
| Subject No. 08 | 79% | 50% | 15% |
| Subject No. 09 | 73% | 45% | 10% |
| Subject No. 10 | 70% | 35% | 20% |
| Subject No. 11 | 65% | 40% | 15% |
| Subject No. 12 | 60% | 35% | 15% |
| Subject No. 13 | 70% | 40% | 10% |
| Subject No. 14 | 80% | 45% | 10% |

Table 1 shows that the number of hot flashes that occurred in GnRHa therapy drops, as a result of the administration of 17α-estradiol, by an average of 10.5% after one week, 53.5% after two weeks and 84.5% after 3 weeks.

No bleeding occurred with the administration of 17α-estradiol.

The values for LH, FSH, prolactin, testosterone, DHEAS, SHBG, estradiol, and progesterone remained low and unchanged.

A withdrawal trial led to an increase in hot flashes, within three weeks back to the starting values before the administration of 17α-estradiol.

No changes in liver values, blood count or electrolytes occurred. There was no change in body weight; for those subjects with borderline RR (n=4, RR around 140/80), the blood pressure dropped to values around 110/70.

The Ca 125 values did not rise.

Endometriosis-specific complaints did not recur during the treatment with 17α-estradiol.

No intracyclic bleeding occurred.

Over one year, for the four subjects monitored, the bone density did not change significantly.

In subjects with fibroid tumors of the uterus, the uterus size decreased to 40±10% of the starting value within three months. This course of the decrease in uterine volume was identical to the course in subjects who did not receive any 17α-estradiol.

The supplementary treatment with 17α-estradiol thus did not influence the effect of the GnRHa therapy.

The results of the study prove that by the administration of the compounds according to the invention, such side effects as hot flashes during and/or after a treatment with analogs or antagonists of gonadotropin releasing hormone (GnRHa therapy) are avoided or reduced.

The hot flashes tripped by GnRHa therapy in the treatment of endometriosis or fibroid tumors of the uterus are effectively eliminated.

In this respect, all the subjects were complaint-free for the duration of the treatment.

In the withdrawal trial, hot flashes occurred again after about 2 to 3 weeks.

The acceptance by the subjects of long-term therapy with GNRH analogs for endometriosis and fibroid tumors of the uterus is markedly improved by additionally administering the preparations according to the invention.

While the invention has been illustrated and described as embodied in pharmaceutical preparations for treating side effects during and/or after GnRHa therapy, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A pharmaceutical preparation for treating side effects during and/or after treatment with analogs or antagonists of gonadotropin releasing hormone, said pharmaceutical preparation containing an effective amount of at least one active ingredient selected from the group comprising 17α-estradiol, chemically modified derivatives of said 17α-estradiol, chemically modified derivatives of 17β-estradiol and chemically modified derivatives of estriol.

2. The pharmaceutical preparation as defined in claim 1, wherein said side effects include hot flashes.

3. A pharmaceutical preparation for treating side effects during and/or after treatment with analogs or antagonists of gonadotropin releasing hormone, said pharmaceutical preparation containing an effective amount of at least one active ingredient selected from the group consisting of 17α-estradiol, ent-17α-estradiol, 4-methyl-estra-1,3,5(10)-triene-1,17α-diol, 4-methylestra-1,3,5(10),6-tetraene-1,17α-diol, 4-methylestra-1,3,5(10),6,8-pentaene-1,17β-diol, ent-estradiol, 4-methylestra-1,3,5(10),6-tetraene-1,17α-diol, 4-methylestra-1,3,5(10),6,8-pentaene-1,17β-diol, 17α-4′-hydroxyphenylmethyl-4-methylestra-1,3,5(10)-triene-1,17β-diol, 17α-4′-hydroxyphenoxymethyl-4-methylestra-1,3,5(10)-triene-1,17β-diol, 17α-4′-hydroxy-thiophenoxymethyl-4-methylestra-1,3,5(10)-triene-1,17β-diol, 17α-4′-dimethylaminophenylmethyl-4-methylestra-1,3,5(10)-triene-1,17β-diol, 17α-3′,5′-dimethyl-4-hydroxyphenylmethyl-4-methylestra-1,3,5(10)-triene-1,17β-diol, 17α-3′,5′-dimethyl-4′-hydroxyphenylmethyl-4-methylestra-1,3,5(10),6-tetraene-1,17β-diol, 17α-4′-hydroxyphenoxylmethyl-4-methylestra-1,3,5(10),6-tetraene-1,17β-diol, 14β,15β-methylene-8-dehydroestradiol, 6-dehydroestriol and 9(11)-dehydroestriol.

4. The pharmaceutical preparation as defined in claim 3, wherein said side effects include hot flashes.
